(19) United States
(12) Patent Application Publication
(10) Pub. No.: US 2021/0307830 A1
(43) Pub. Date: Oct. 7, 2021

(54) Method and Apparatus for Providing Procedural Information Using Surface Mapping

(71) Applicant: TransEnterix Surgical, Inc., Morrisville, NC (US)

(72) Inventors: Kevin Andrew Hufford, Cary, NC (US); Mohan Nathan, Raleigh, NC (US)

(21) Appl. No.: 16/018,039

(22) Filed: Jun. 25, 2018

Related U.S. Application Data
(60) Provisional application No. 62/624,143, filed on Jan. 31, 2018.

(51) Int. Cl. A61B 34/10; A61B 34/32; A61B 17/3207; G16H 20/40

(52) U.S. Cl. CPC A61B 34/10; A61B 34/32; A61B 17/3207; A61B 1/0661; A61B 2034/104; A61B 2034/105; G16H 20/40

(57) ABSTRACT

In a system and method for assessing tissue excision comprise, first 3-dimensional data is acquired for a surgical region of interest from which tissue is to be excised, the first data defining initial geometry of tissue in the region of interest. A desired excision parameter, such as depth or shape, is determined and tissue is excised from the region of interest. Second 3-dimensional data for the region of interest is then acquired, the second scan data defining post-excision geometry of the tissue in the region of interest. The first and second data is compared to determine whether the desired excision parameter has been reached. The 3-dimensional data may be scan data acquired using a 3D or 2D endoscope, and/or it may be derived from kinematic data generated as a result of moving an instrument tip over the region of interest.

### BACKGROUND

Various surface mapping methods exist that allow the topography of a surface to be determined. One type of surface mapping method is one using structured light. Structured light techniques are used in a variety of contexts to generate three-dimensional (3D) maps or models of surfaces. These techniques include projecting a pattern of structured light (e.g. a grid or a series of stripes) onto an object or surface. One or more cameras capture an image of the projected pattern. From the captured images the system can determine the distance from the camera to the surface at various points, allowing the topography/shape of the surface to be determined.

In the performance of a surgical procedure, sometimes it is necessary to excise tissue. Advanced imaging and measurement techniques provide the means of greater assurance that the procedural goals are achieved.

The methods described herein may be used with surgical robotic systems. There are different types of robotic systems on the market or under development. Some surgical robotic systems use a plurality of robotic arms. Each arm carries a surgical instrument, or the camera used to capture images from within the body for display on a monitor. Other surgical robotic systems use a single arm that carries a plurality of instruments and a camera that extend into the body via a single incision. Each of these types of robotic systems uses motors to position and/or orient the camera and instruments and to, where applicable, actuate the instruments. Typical configurations allow two or three instruments and the camera to be supported and manipulated by the system. Input to the system is generated based on input from a surgeon positioned at a master console, typically using input devices such as input handles and a foot pedal. Motion and actuation of the surgical instruments and the camera is controlled based on the user input. The system may be configured to deliver haptic feedback to the surgeon at the controls, such as by causing the surgeon to feel resistance at the input handles that is proportional to the forces experienced by the instruments moved within the body. The image captured by the camera is shown on a display at the surgeon console. The console may be located patient-side, within the sterile field, or outside of the sterile field.

### BRIEF DESCRIPTION OF THE DRAWINGS

FIG. 1 illustrates a body organ and a tumor to be excised from that organ.

FIG. 2(a) shows a representation of a scan of an organ prior to removal of a tumor from that organ; FIG. 2(b) shows a representation of a scan of the organ of FIG. 2(b) following removal of the tumor, which an overly depicting comparative information generated from the pre- and post-excision scans.

FIG. 3 schematically illustrates a method of using scans taken prior to and after a procedural step to determine whether procedural objectives have been achieved

### DETAILED DESCRIPTION

This application describes the use of surface mapping techniques to aid the surgeon in determining whether a desired step in a surgical procedure has been achieved. The described methods are particularly useful for procedures requiring the excision of tissue. Positional data from the surgical site provides valuable comparative information that may be used. This positional data may be obtained from a wide area scan of the surgical site, or from a scan of a particular region of interest, or any combination thereof.

The described methods may be performed using a robotic surgical system, although they can also be implemented without the use of surgical robotic systems.

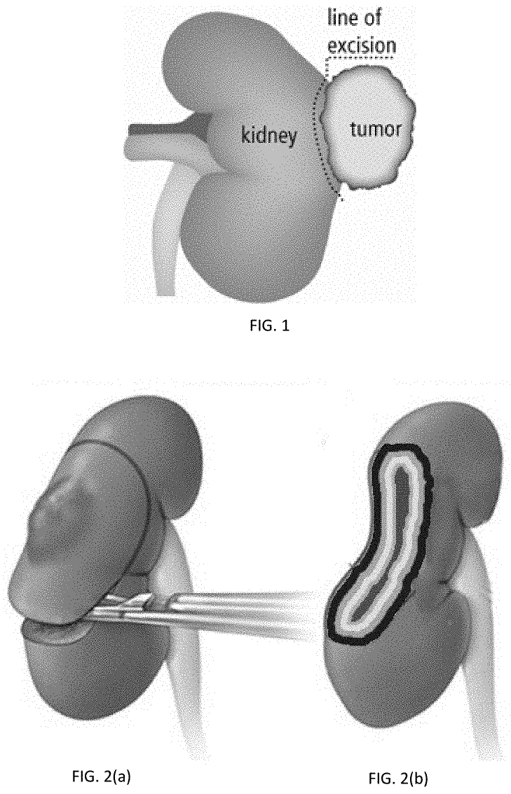

An exemplary method will be performed in the context of a procedure for the excision of a tumor in a partial nephrectomy. For the removal of the tumor, a surgeon typically seeks to excise both the tumor and margins of a certain depth around the tumor. The surgeon will thus determine a path for the excision instrument, or a certain excision depth, or other parameters that will produce the appropriate margin. See FIG. 1.

In accordance with the disclosed method, prior to a partial nephrectomy, an initial scan is captured of the kidney and tumor to provide the initial 3-dimensional position and shape information for these structures as shown in FIG. 2(a). Following the excision, a second scan of the area is captured as shown and the data from the two images is compared. An image may be displayed to the surgeon to include information that aids the surgeon in assessing the excision. For example, FIG. 2(b) shows an image of the region that has been excised, with a colored overlay that provides feedback to the surgeon. Colors Represented in this view may be based on actual deviation from the original scan, or may alternatively be based on achievement of the original planned shape, or originally-defined depth.

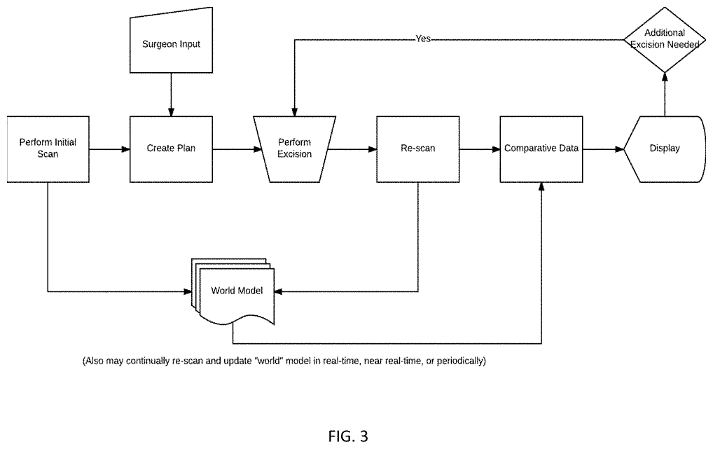

The comparative data thus provides information that allows the surgeon to determine that the appropriate depth has been achieved, or to conclude additional excision is needed. The method is depicted schematically in FIG. 3.

As one example, if the tumor and selected margin has been determined to be 3 cm deep, comparing the scan data may result in overlays that allows the surgeon to see whether the desired 3 cm depth was achieved by the excision.

In some cases, the 3-dimensional pre-excision and post-excision scans may provide a comparative data set for a surface or series of points rather than just a single point or depth.

Because of the nature of the soft-tissue environment of abdominal surgery, in some cases, registration is performed between the 3D data sets captured before and after the excision. This may use anatomical landmarks, surface curvature, visual texture, or other means or combinations of means to determine that the changes are due to the procedure, and not simply deflections or repositioning of soft tissue structures. A soft tissue deformation model such as one using finite-element techniques may also be constructed and may be updated periodically to accurately track deformations.

This 3-dimensional data may be gathered using various scanning techniques, including stereoscopic information from a 3D endoscope, structured light measured by a 3D endoscope, structured light measured by a 2D endoscope, or a combination thereof.

During the capture of a scan, feedback may be given to the user about the suitability of a scan/the comprehensiveness of a scan. On-screen prompts may provide overlays about the scan coverage, provide cueing inputs for a scan, and/or walk the user through a series of steps.

In some implementations, the robotic surgical system may perform an autonomous move/series of moves/sequence of moves to scan around a wide view, a smaller region, or a particular region of interest. This scan may be pre-programmed or may be selected or modified by the user.

In some implementations, the robotic surgical system may use kinematic knowledge from the surgical robotic system to provide information about the relative positions of the initial and final positions of the surgical instrument robotically controlled to perform the excision. In this use case, the surgeon (or robotic system) may cause the robotically-moved surgical instrument to touch a given surface using the surgical instrument, and the pose of the instrument tip (position and orientation in Cartesian space) may be recorded. After the excision is be performed a post-excision measurement is taken. The instrument is used to touch the excised surface, providing pose information relative to that of the previous pose. This process may be carried out at a certain point or a series of points, which may be used to define a plane or a surface.

In some implementations, the depth from the original surface may be continuously displayed as an overlay on the screen. This may be, for example, but not limited to, in the corner of the screen, or as an unobtrusive overlay near the laparoscopic tool tip.

In some implementations, the robotic surgical system may perform the scan(s) and/or excisions/treatment autonomously or semi-autonomously, with the surgeon providing an initiation and/or approval before and/or after of all or certain steps.

Co-pending U.S. application Ser. No. 16/010,388 filed Jun. 15, 2018, describes creation, and use of a “world model”, or a spatial layout of the environment within the body cavity, which includes the relevant anatomy and tissues/structures within the body cavity that are to be avoided by surgical instruments during a robotic surgery. The systems and methods described in this application may provide 3D data for the world model or associated kinematic models in that (see for example FIG. 5 of that application) type of system and process. See, also, FIG. 3 herein, in which the world view is updated based on the pre-excision and post-excision scans, and informs the comparison of the data.

This technology may use the multiple vantage point scanning techniques from co-pending U.S. application Ser. No. 16/______, filed Jun. 25, 2018, entitled Method and Apparatus for Providing Improved Peri-operative Scans, (Ref: TRX-16210).

All applications referred to herein are incorporated herein by reference.

1-12. (canceled)

13. A method of assessing tissue excision, comprising:
(a) acquiring first 3-dimensional data for a surgical region of interest from which tissue is to be excised, the first data defining initial geometry of tissue in the region of interest;
(b) determining a desired excision parameter;
(c) excising tissue from the region of interest;
(d) acquiring second 3-dimensional data for the region of interest following the step of excision tissue, the second data defining post-excision geometry of the tissue in the region of interest;
(e) determining, based on a comparison of the first and second data, whether the desired excision parameter has been reached, and repeating steps (a), (c), (d) and (e) until the desired excision parameter has been reached.

14. The method of claim 13, wherein the desired excision parameter is input into a surgical robotic system and steps (a), (b), (c) and (e) are performed autonomously by the surgical robotic system.

15. The method of claim 14, wherein step (e) is performed using additional data from sensors in the robotic system.

16. The method of claim 14, wherein the method is semiautonomous with surgeon approving plan and providing a check that plan was achieved/result is acceptable.

17. The method of claim 13, in which the first data is at least partially generated by positioning an instrument tip on the surface of the region of interest and determining the location or pose of the instrument tip, and the second data is generated by positioning the instrument tip on the excised surface of the region of interest and determining the location or pose of the instrument tip.

18. The method of claim 13, wherein at least the first or second 3-dimensional data is 3-dimensional scan data acquired using a 3-dimensional endoscope system.

19. The method of claim 18, wherein at least the first or second 3-dimensional data is 3-dimensional scan data acquired using a 3-dimensional endoscope system in combination with a structured light source.

20. The method of claim 13, wherein at least the first or second 3-dimensional data is 3-dimensional scan data acquired by capturing images using a 2-dimensional endoscope while moving the 2-dimensional endoscope, to create a 3-dimensional model.

21. The method of claim 13, wherein at least the first or second 3-dimensional data is 3-dimensional scan data acquired by capturing images using a 2-dimensional endoscope in combination with a structured light source.

22. The method of 13, further comprising:
providing feedback relating to the depth of the excision based on a comparison of the first and second scan data.

23. The method of claim 22, wherein the step of providing feedback includes displaying on a display display an image of the region of interest with an overlay representing comparative information resulting from a comparison of the first and second scans.

24. The method of claim 23, wherein the image displays the region of interest following excision of tissue and the overlay represents data relating to three dimensional properties of the excised tissue.

24. (canceled)

25. The method of claim 22, wherein the feedback includes a display of an image of the post-excision region of interest with a colored overlay representing the spatial deviation of the excised surface from a prescribed depth.

26. The method of claim 22, wherein the feedback includes a display of an image of the post-excision region of interest with a colored overlay identifying the spatial deviation of the position of the excised surface compared with the position of the tissue surface prior to excision.
